(19) United States
(12) Patent Application Publication
(10) Pub. No.: US 2012/0197319 A1
(43) Pub. Date: Aug. 2, 2012

(54) INFLATABLE BONE TAMP WITH ADJUSTABLE WORKING LENGTH

(75) Inventors: Bryan J. Auyoung, Santa Clara, CA (US); Warren C. Sapida, Sunnyvale, CA (US)

(73) Assignee: KYPHON SARL, Neuchatel (CH)

(21) Appl. No.: 13/014,939

(22) Filed: Jan. 27, 2011

(51) Int. Cl. A61B 17/56

(52) U.S. Cl. 606/86 R

(57) ABSTRACT

An inflatable bone tamp for performing a minimally invasive surgical procedure includes an extension controller for adjusting the relative position between the inner shaft and the outer shaft, thereby allowing the working length of the inflatable structure (e.g., balloon) to be adjusted and set prior to (and/or optionally during) use. The extension controller allows for customization of the inflatable bone tamp performance characteristics to enhance surgical effectiveness for a given physical condition.

### FIELD OF THE INVENTION

The invention relates to a system and method for performing a surgical procedure, and in particular, to an inflatable device that exhibits a presettable balloon length.

### BACKGROUND OF THE INVENTION

A minimally invasive procedure is a medical procedure that is performed through the skin or an anatomical opening. In contrast to an open procedure for the same purpose, a minimally invasive procedure will generally be less traumatic to the patient and result in a reduced recovery period.

However, there are numerous challenges that minimally invasive procedures present. For example, minimally invasive procedures are typically more time-consuming than their open procedure analogues due to the challenges of working within a constrained operative pathway. In addition, without direct visual feedback into the operative location, accurately selecting, sizing, placing, and/or applying minimally invasive surgical instruments and/or treatment materials/devices can be difficult.

For example, for many individuals in our aging world population, undiagnosed and/or untreatable bone strength losses have weakened these individuals' bones to a point that even normal daily activities pose a significant threat of fracture. In one common scenario, when the bones of the spine are sufficiently weakened, the compressive forces in the spine can cause fracture and/or deformation of the vertebral bodies. For sufficiently weakened bone, even normal daily activities like walking down steps or carrying groceries can cause a collapse of one or more spinal bones. A fracture of the vertebral body in this manner is typically referred to as a vertebral compression fracture. Other commonly occurring fractures resulting from weakened bones can include hip, wrist, knee and ankle fractures, to name a few.

Fractures such as vertebral compression fractures often result in episodes of pain that are chronic and intense. Aside from the pain caused by the fracture itself, the involvement of the spinal column can result in pinched and/or damaged nerves, causing paralysis, loss of function, and intense pain which radiates throughout the patient's body. Even where nerves are not affected, however, the intense pain associated with all types of fractures is debilitating, resulting in a great deal of stress, impaired mobility and other long-term consequences. For example, progressive spinal fractures can, over time, cause serious deformation of the spine (“kyphosis”), giving an individual a hunched-back appearance, and can also result in significantly reduced lung capacity and increased mortality.

Because patients with these problems are typically older, and often suffer from various othersignificant health complications, many of these individuals are unable to tolerate invasive surgery. Therefore, in an effort to more effectively and directly treat vertebral compression fractures, minimally invasive techniques such as vertebroplasty and, subsequently, kyphoplasty, have been developed. Vertebroplasty involves the injection of a flowable reinforcing material, usually polymethylmethacrylate (PMMA—commonly known as bone cement), into a fractured, weakened, or diseased vertebral body. Shortly after injection, the liquid filling material hardens or polymerizes, desirably supporting the vertebral body internally, alleviating pain and preventing further collapse of the injected vertebral body.

Because the liquid bone cement naturally follows the path of least resistance within bone, and because the small-diameter needles used to deliver bone cement in vertebroplasty procedure require either high delivery pressures and/or less viscous bone cements, ensuring that the bone cement remains within the already compromised vertebral body is a significant concern in vertebroplasty procedures. Kyphoplasty addresses this issue by first creating a cavity within the vertebral body (e.g., with an inflatable balloon) and then filling that cavity with bone filler material. The cavity provides a natural containment region that minimizes the risk of bone filler material escape from the vertebral body. An additional benefit of kyphoplasty is that the creation of the cavity can also restore the original height of the vertebral body, further enhancing the benefit of the procedure.

Conventional inflatable bone tamps (IBTs) used in kyphoplasty procedures incorporate balloon catheters that are constructed using two coaxial catheters, with the distal ends of the outer and inner catheters being coupled to the proximal and distal end regions, respectively, of the balloon. The position of the inner catheter relative to the outer catheter, and in particular, the distance the distal end of the inner catheter extends beyond the distal end of the outer catheter, defines an operating length for the balloon.

For many applications, such as use in a kyphoplasty procedure, the particular size, condition, and/or position of the target surgical location can mandate the use of an inflatable bone tamp having a specific balloon length. Typically, multiple inflatable bone tamps of varying balloon lengths are provided to address this need for different balloon lengths. However, this can undesirably increase procedure costs, and in addition, an inflatable bone tamp having the ideal balloon length may still not be available from among the premade products.

Accordingly, it is desirable to provide surgical tools and techniques that enable the implementation and use of an inflatable bone tamp having an adjustable balloon length.

### SUMMARY OF THE INVENTION

By providing an inflatable bone tamp that incorporates a position controller for an inner shaft coupled to a distal tip of the balloon, a customized balloon working length can be set for the balloon as desired by the user (surgeon).

In one embodiment, an inflatable bone tamp can include outer shaft, an inner shaft disposed within the outer shaft, an inflatable structure having proximal and distal ends coupled to the distal ends of the outer shaft and the inner shaft, respectively, and an extension controller. The extension controller adjusts and sets the relative position between the distal ends of the inner and outer shafts, thereby defining the working length (i.e., initial length) of the inflatable structure. In various embodiments, the extension controller can include a friction mechanism (e.g., pull rollers), a screw mechanism, and/or a gear mechanism for advancing/retracting the inner shaft, and a ratchet, latch, clamp, and/or other securing mechanism for fixing the relative position between the inner and outer shafts.

In some embodiments, the extension controller can also include a sealing element for preventing leakage of inflation fluid around the inner shaft, while still allowing movement of the inner shaft relative to the outer shaft, such as a Tuohy-Borst connector, a flexible gasket, and o-rings mounted on the inner shaft, among others. In various other embodiments, the extension controller can also include a rotation controller for rotating the inner shaft relative to the outer shaft,

In some embodiments, the inner shaft can be a catheter (e.g., polyurethane, polyethylene, and/or nylon) and/or a stainless steel and/or nitinol wire, and/or any other material, optionally with features for engaging with the extension controller to facilitate movement control. Similarly, in various embodiments, the inflatable structure can be formed from any material, and can take any desired configuration (e.g., single chamber, multi-lobe, multi-balloon, etc).

In various other embodiments, a surgical procedure such as kyphoplasty can be performed by creating an access path (e.g., using a cannula), setting the working length for the inflatable structure of an inflatable bone tamp by adjusting and fixing the relative position between the inner and outer shafts of the inflatable bone tamp, inserting the inflatable bone tamp into a target bone (e.g., a fractured vertebra) via the access path, inflating the bone tamp create a cavity in cancellous bone and optionally restoring the original cortical bone profile (e.g., restore vertebral body height), deflating and removing the inflatable bone tamp, and then filling the cavity with bone filler material to support the treated bone. In some embodiments, the relative position between the inner and outer shafts may be adjusted during inflation.

In another embodiment, a surgical system for treating bone can include one or more inflatable bone tamps incorporating extension controllers for setting the working length of the inflatable structures of those bone tamps by adjusting the relative positions of the inner and outer shafts of the bone tamps. The surgical system can further include additional equipment for performing a surgical procedure using the inflatable bone tamp(s) (e.g., one or more cannulas sized to accept the inflatable bone tamps, access tools such as drills, guide wires, obturators, trocars, and/or curettes) and/or instructions for performing the surgical procedure using the one or more inflatable bone tamps.

As will be realized by those of skilled in the art, many different embodiments of an inflatable bone tamp incorporating an inner shaft having an extension controller for setting working length, and systems, kits, and/or methods of using such an inflatable bone tamp according to the present invention are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIGS. 1A-1B show an exemplary inflatable bone tamp incorporating an extension controller for adjusting the working length of the inflatable structure of the balloon tamp.

FIGS. 2A-2B show exemplary embodiments of an extension controller for the inflatable bone tamp of FIGS. 1A-1B.

FIGS. 3A-3B show exemplary inflatable structures for the inflatable bone tamp of FIGS. 1A-1B.

FIGS. 4A-4H show an exemplary kyphoplasty procedure that incorporates the inflatable bone tamp of FIGS. 1A-1B.

FIG. 5 shows a kit that includes the inflatable bone tamp of FIGS. 1A-1B.

FIG. 6 shows a flow diagram for an exemplary surgical procedure that makes use of the inflatable bone tamp of FIGS. 1A-1B.

### DETAILED DESCRIPTION

By providing an inflatable bone tamp that incorporates a position controller for an inner shaft coupled to a distal tip of the balloon, a customized balloon length can be set for the balloon as desired by the user (surgeon).

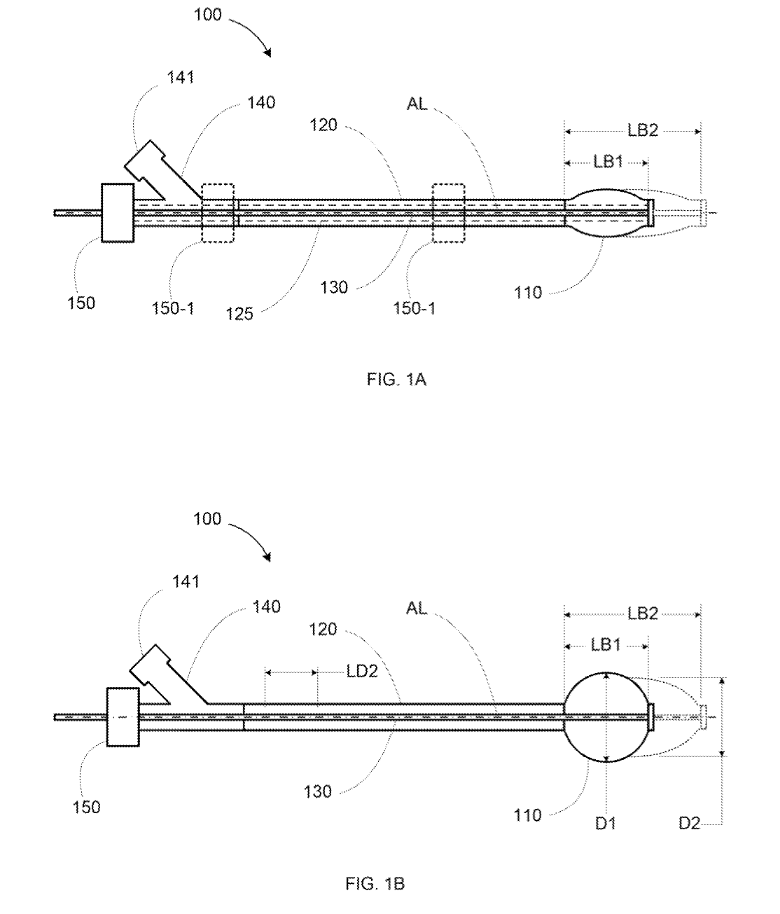

FIG. 1A shows an embodiment of an inflatable bone tamp 100 that includes an inflatable structure 110, an outer shaft 120, an inner shaft 130 disposed at least partially within outer shaft 120, a connector 140, and an extension controller 150. The proximal end regions of inner shaft 130 and outer shaft 120 are coupled to connector 140, while the distal end regions of inner shaft 130 and outer shaft 120 are coupled to the distal and proximal end regions, respectively, of inflatable structure 110.

In one embodiment, inflatable structure 110 can be inflated through a lumen formed between outer shaft 120 and inner shaft 130 (e.g., using inflation fluid delivered via connector 140). In another embodiment, inner shaft 130 can itself be a catheter for delivering the inflation fluid to inflatable structure 110. And in another embodiment, inflatable bone tamp 100 can include an optional additional inner catheter 125 (indicated by dashed lines) for defining an inflation fluid flow path (either between catheter 125 and outer shaft 120, between catheter 125 and inner shaft 130, or within catheter 125).

The distal end of inner shaft 130 extends beyond the distal end of outer shaft 120, thereby defining an operating length LB for inflatable structure 110. This operating length can be adjusted by extension controller 150, which interfaces with inner shaft 130 to set the relative extension of inner shaft 130 beyond the distal end of outer shaft 120. Note that while extension controller 150 is depicted as being positioned at the proximal end of inflatable bone tamp 100 and adjacent to connector 140 for exemplary purposes, in various other embodiments, extension controller 150 can be positioned anywhere along inflatable bone tamp 100, as indicated by the dotted outlines of extension controllers 150-1 and 150-2.

The adjustment capability provided by extension controller 150 defines the working length of inflatable structure 110—i.e., the length of inflatable structure in its inflated, non-distended state. For example, if inner shaft 130 is in a relatively retracted position with respect to outer shaft 120, inflatable structure 110 will have a shorter working length LB1 than if inner shaft 130 is in a relatively extended position with respect to outer shaft 120 (e.g., providing inflatable structure 110 with a longer working length LB2). Note that depending on the construction of inner shaft 130 (e.g., material extensibility/compliance, features, etc.) and the inflation characteristics of inflatable structure 110 (e.g., material, shape, operating pressure, etc.), the maximum length achieved by inflatable structure 110 during use may be slightly greater than the working length set by extension controller 150.

The inflation profile of inflatable structure 110 can be significantly affected by the working length set by extension controller 150. Typically, a shorter working length will result in more radial growth for a given inflation volume than would be achieved with a longer working length. An example of this disparity is depicted in FIG. 1B, wherein inflation of inflatable structure 110 when set to have a shorter working length (LB1) results in a maximum diameter D1 that is greater than a maximum diameter D2 achieved when inflatable structure 110 is set to a longer working length (LB2). The larger radial diameter inflation profile (due to length LB1) could, for example, be desirable in situations where significant lifting is required (e.g., an acute VCF), whereas the longer, flatter inflation profile (due to length LB2) could be beneficial where localized pressure hotspot minimization is prudent (e.g., weakened endplates in a fractured vertebra). Various other usages and benefits that accrue from the controllable length of inflatable bone tamp 100 will be readily apparent.

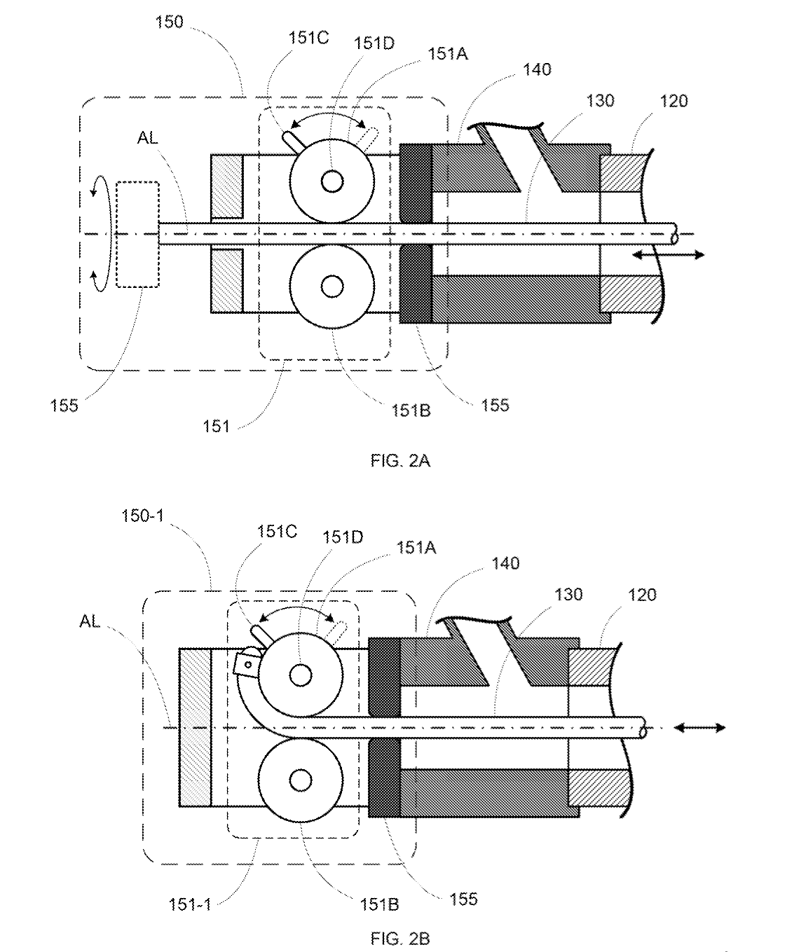

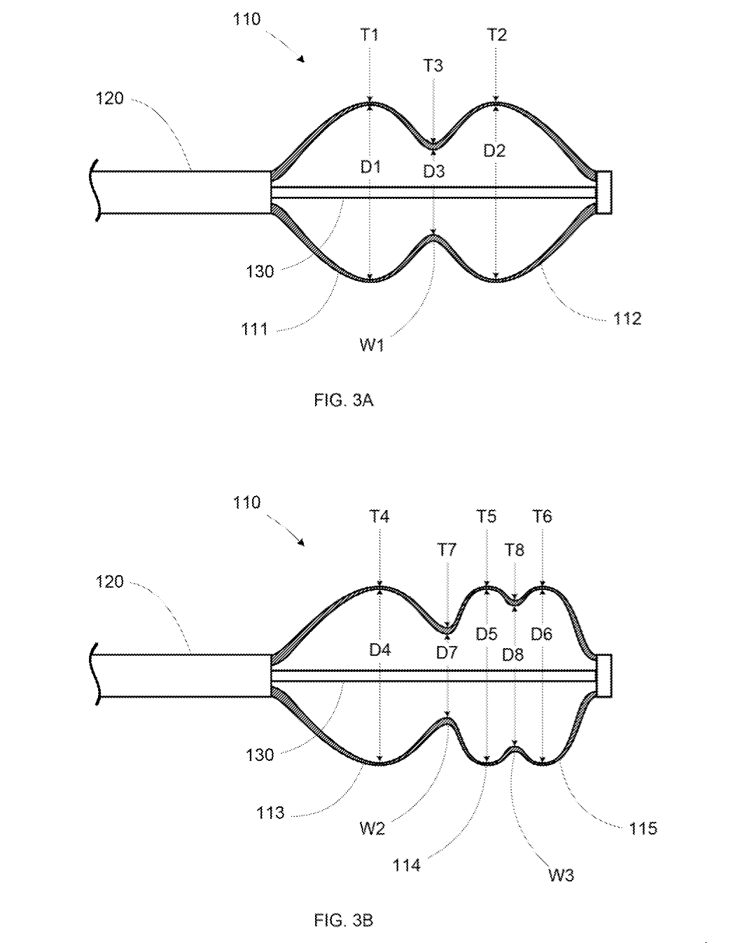

Extension controller 150 can use any mechanism for adjusting and setting the position of inner shaft 130 relative to outer shaft 120. For example, FIG. 2A shows an exemplary embodiment of extension controller 150 that includes a drive mechanism 151 formed by rotary driver elements 151A and 151B. Rotary driver elements 151A and 151B are engaged with inner shaft 130 such that rotation of driver elements 151A and 151B (e.g., in response to user movement of an optional actuator 151C) adjusts the longitudinal position of inner shaft 130 (i.e., the position of inner shaft 130 relative to outer shaft 120 along longitudinal axis AL). A locking mechanism 151D can then be used to fix the position of inner shaft 130. Locking mechanism 151D can be a ratchet mechanism, a clamp, a releasable latch, or any other mechanism for maintaining the position set by extension drive mechanism 151.

Note that in some embodiments, locking mechanism 151D can allow inner shaft 130 to be set at specific predetermined positions that correspond to specific lengths for inflatable structure 110 (e.g., a latching mechanism that engages when inner shaft 130 is in one of three positions that correspond to three specific lengths for inflatable structure 110 determined to be most generally applicable for the bone structure conditions expected for a given procedure). In other embodiments, locking mechanism 151D can allow for more length variability, either in discrete increments (e.g., a ratchet) or continuously (e.g., a friction fit and/or clamp).

In some embodiments, extension controller can further include a sealing element 155 that allows for passage, movement, and/or manipulation of inner shaft 130 without allowing leakage of inflation fluid delivered via connector 140 and/or outer shaft 120 to inflatable structure 110 (not shown). For example, sealing element 155 can be an elastomeric gasket, a Tuohy-Borst connector, an o-ring(s) seated in inner shaft 130, or any other mechanism providing leak-resistant relative motion capabilities.

Note that in various embodiments, drive mechanism 151 can incorporate a friction drive, such that driver element 151A and/or 151B simply press against inner shaft 130 and rotate to advance/retract inner shaft 130 (e.g., pull rollers). In various other embodiments, driver element 151A and/or 151B can be a gear (e.g., spur gear, helical gear, worm wheel gear, rack gear, etc.) that engages with notches, grooves, threads, or any other features on inner shaft 130.

In various other embodiments, extension drive mechanism 151 can further include an optional rotation controller 155 that rotates inner shaft 130 with respect to outer shaft 120. This can allow inflatable structure 110 to be wrapped around inner shaft 130 to facilitate positioning and/or removal of inflatable bone tamp 100 in confined spaces. Note that while depicted as a simple knob attached to inner shaft 130 for exemplary purposes, various other embodiments will be readily apparent, including having extension controller 150 itself rotate to rotate inner shaft 130.

In general, inner shaft 130 will be a generally rigid element that is longitudinally inextensible (e.g., stainless steel or nitinol wire/rod) or minimally longitudinally extensible (e.g., polyurethane or nylon catheter), or a combination of various materials. Typically, such embodiments of inner shaft 130 would be substantially rigid as well, but in some embodiments, inner shaft 130 can be a flexible element.

For example, FIG. 2B shows an alternative embodiment of inner shaft 130 that exhibits flexibility while maintaining a desired degree of longitudinal inextensibility (e.g., a push-pull cable or nitinol wire, among others). In FIG. 2B, inner shaft 130 is wrapped/unwrapped around driver element 151A to retract/extend inner shaft 130. Various other embodiments will be readily apparent.

Returning to FIGS. 1A and 1B, note that while inflatable structure 110 is depicted as a simple, single lobed balloon for exemplary purposes, in various other embodiments, inflatable structure 110 can take any form that would benefit from the length adjustment capability provided by extension controller 150.

For example, FIG. 3A shows an exemplary “peanut-shaped” balloon what that includes two lobes 111 and 112 joined at a narrowed waist W1 (i.e., maximum non-distended (i.e., non-stretched) diameters D1 and D2 of lobes 111 and 112, respectively, are greater than the minimum diameter D3 of waist W1). The peanut shape can beneficially result in a more ovoid inflation profile that can enhance the performance of the inflatable bone tamp. The inflation profile can be further enhanced by controlling the wall thickness profile of inflatable structure 110 (e.g., by forming waist W1 to have a maximum thickness T3 that is greater than the minimum thickness T1 and T2 of lobes 111 and 112, respectively.

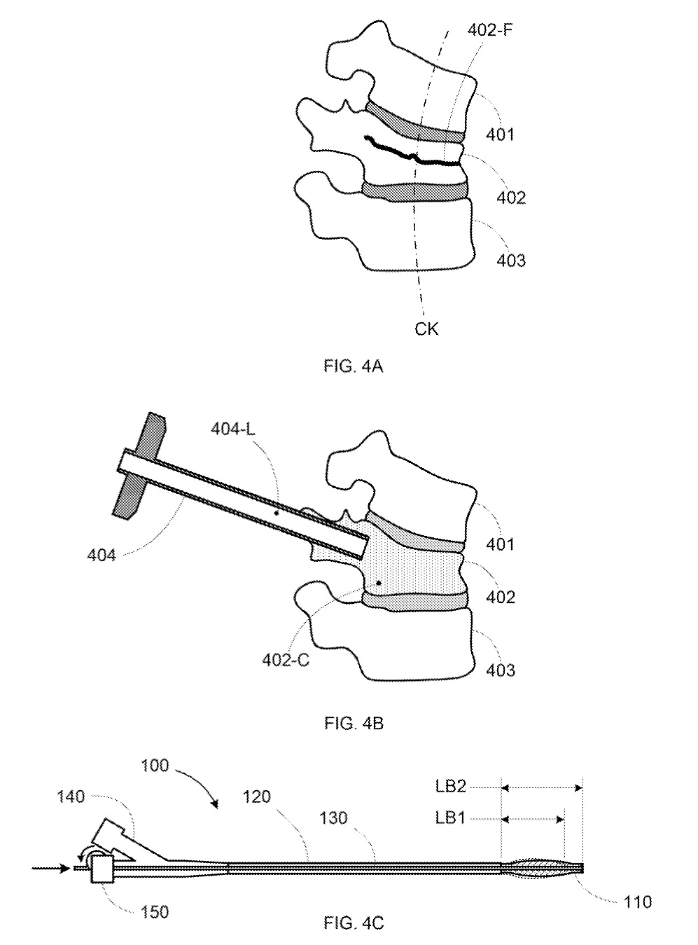

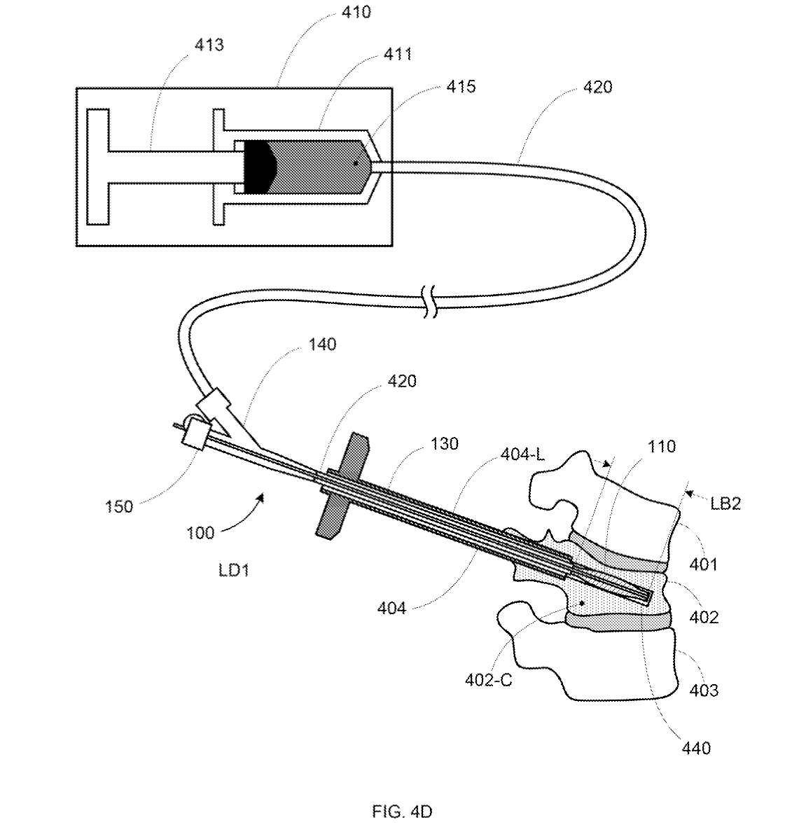

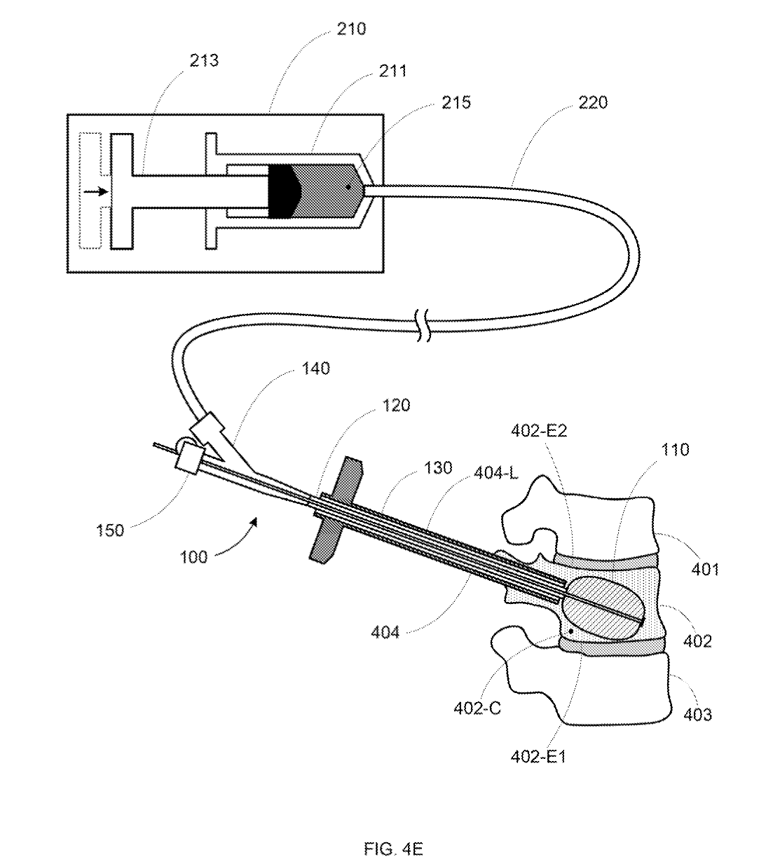

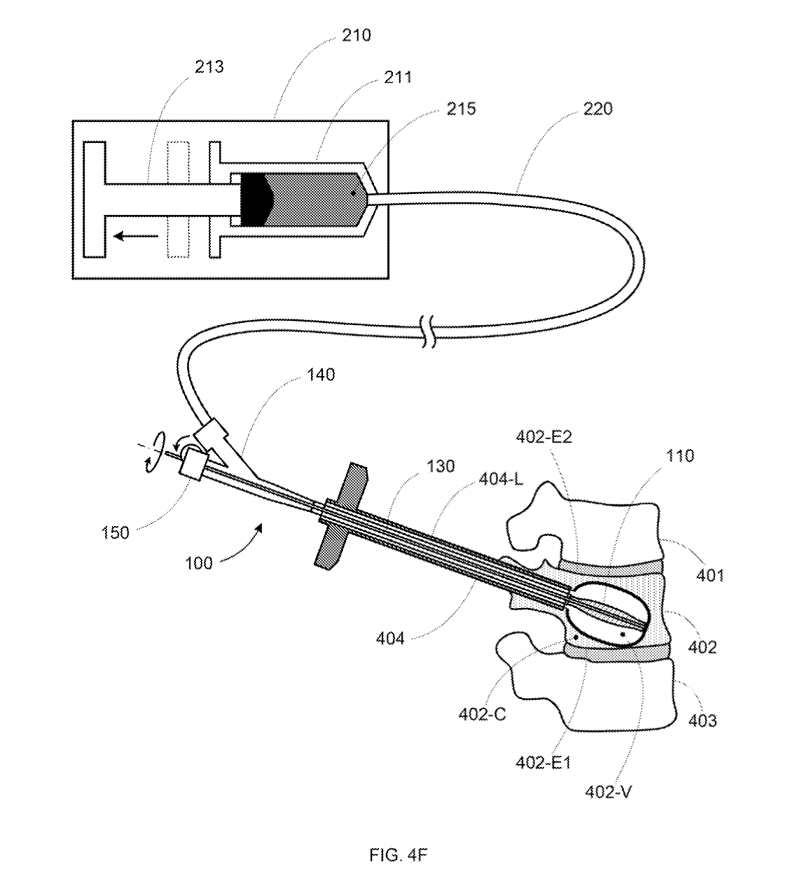

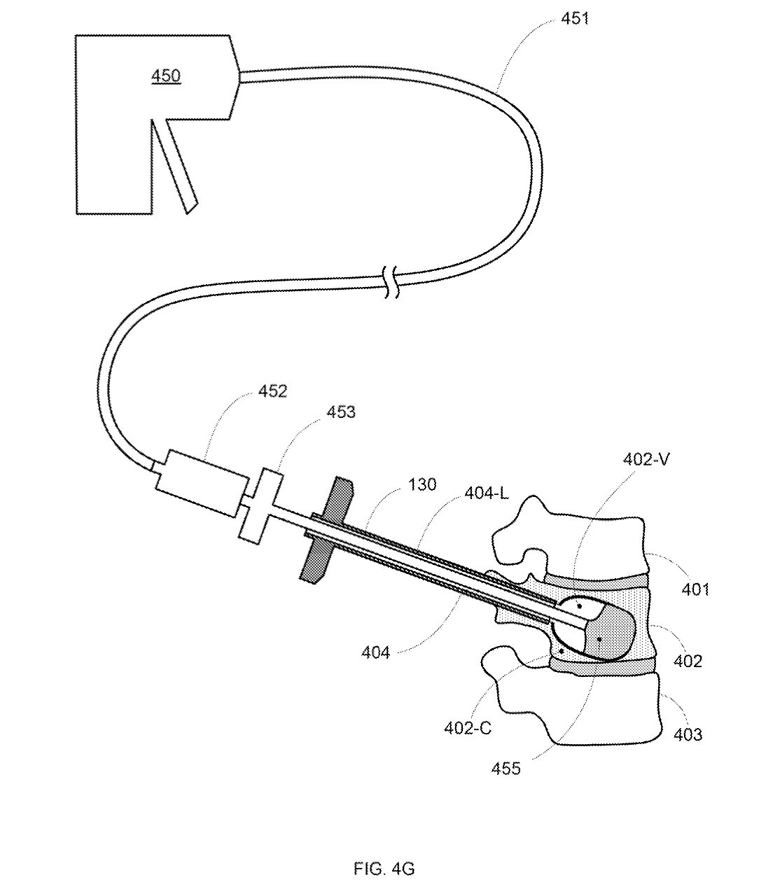

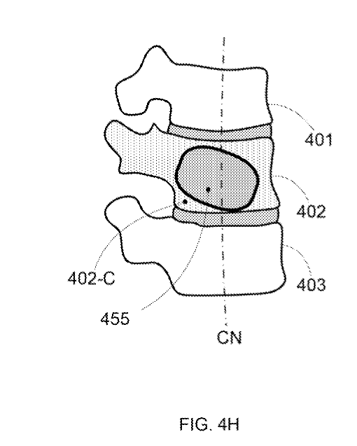

FIG. 3B shows another exemplary balloon construction for inflatable structure 110 that includes lobes 113 and 114 joined at a narrowed waist W2 (i.e., maximum non-distended diameters D4 and D5 greater than the minimum diameter D7 of waist W2), and an additional lobe 115 joined to lobe 114 at a second narrowed waist W3 (i.e., maximum non-distended diameters D5 and D6 greater than the minimum diameter D8 of waist W3). The multi-lobe configuration shown in FIG. 4B can result in an inflation profile exhibiting an outwardly tapering inflation profile, such that the maximum distended diameter of inflatable structure 110 occurs towards the distal end of inflatable structure 110, which can beneficially enhance performance during certain procedures, such as kyphoplasty. Various other balloon configurations will be readily apparent (e.g., multi-chambered, multi-balloon, and/or various shapes, among others).

FIGS. 4A-4H show an exemplary kyphoplasty procedure using an inflatable bone tamp incorporating an inflatable structure with an adjustable working length. FIG. 4A shows a portion of a human vertebral column having vertebrae 401, 402, and 403. Vertebra 402 has collapsed due to a vertebral compression fracture (VCF) 402-F that could be the result of osteoporosis, cancer-related weakening of the bone, and/or physical trauma. The abnormal curvature CK of the spine caused by VCF 402-F can lead to severe pain and further fracturing of adjacent vertebral bodies.

FIG. 4B shows a cannula 404 being positioned next to the target surgical location, which in this case is the cancellous bone structure 402-C within fractured vertebra 402. In this manner, a percutaneous path to vertebra 402 is provided via an interior lumen 404-L of cannula 404. Typically, cannula 404 is docked into the exterior wall of the vertebral body (using either a transpedicular or extrapedicular approach) using a guide needle and/or dissector, after which a drill or other access tool (not shown) is used to create a path further into the cancellous bone 402-C of vertebra 402. However, any other method of cannula placement can be used to position cannula 404.

Meanwhile, as shown in FIG. 4C, an inflatable bone tamp 100 (as described with respect to FIGS. 1A-1B) is adjusted as desired by the user (e.g., surgeon). Specifically, as described above, extension controller 150 is used to adjust and set the length of the inflatable structure 110. For example, as indicated in FIG. 4C, extension controller 150 can be actuated (as indicated by the curved arrow) to set the working length of inflatable structure 110 at a length LB2, from a shorter length LB1. Various other length adjustments will be readily apparent.

Then in FIG. 4D, inflatable bone tamp 100 is placed into cannula 404. Inflatable bone tamp 100 is coupled to an inflation mechanism 410 by a flow channel 420 (e.g., flexible tubing). For exemplary purposes, inflation mechanism 410 is depicted as a syringe having a plunger 413 for expressing inflation fluid 415 (e.g., saline solution, air, contrast solution, or any other fluid) from a barrel 411. Note that in various other embodiments, inflation mechanism 410 can be any system for delivering inflation, such as a syringe, pump, or compressed gas system, among others. Furthermore, in various other embodiments, inflation mechanism 410 can be directly connected to inflatable bone tamp 100.

As inflation mechanism 410 is actuated to drive inflation fluid 415 into inflatable structure 110, inflatable structure 110 begins to expand within fractured vertebra 402. For example, in the embodiment shown in FIG. 4E, a force is applied to drive plunger 413 through barrel 411, thereby expressing inflation fluid 415 through flow channel 420, connector 140, outer shaft 120 and/or inner shaft 130, and into inflatable structure 110. The resulting expansion of inflatable structure 110 initially compresses the surrounding cancellous bone 402-C to begin creating a cavity within vertebra 402, and can also push apart the harder endplates 402-E1 (inferior) and 402-E2 (superior) of vertebra 402 apart to restore the height of fractured vertebra 402.

In many instances, the likelihood of high quality cavity creation and/or height restoration in vertebra 402 can be increased through the appropriate setting of the working length of inflatable structure 110, as described with respect to FIG. 4C. Note that in some embodiments, the preset working length LB2 can be further adjusted during inflation of inflatable structure 110 (e.g., disengaging locking mechanism 151D, described above, adjusting the position of inner shaft 130 relative to outer shaft 120, and then re-engaging locking mechanism 151D, as described above with respect to FIG. 2A).

Once inflatable structure 110 has been expanded to a desired volume and/or a desired height restoration has been achieved in vertebra 402, inflatable structure 110 is deflated, leaving a well-defined cavity 402-V, as shown in FIG. 4F. Note that in some embodiments, extension controller 150 can at this point be used to further extend inner shaft 130 with respect to outer shaft 120 (as indicated by the curved directional arrow), thereby lengthening inflatable structure 110 (now deflated) to reduce the overall diameter of inflatable structure 110 to facilitate removal through cannula 404. In various other embodiments, extension controller 150 can alternatively or additionally facilitate removal of inflatable bone tamp 100 from cannula 404 by rotating inner shaft 130 with respect to outer shaft 120 (as indicated by the circular directional arrow), thereby wrapping inflatable structure 110 around inner shaft 130.

Inflatable bone tamp 100 can then be removed from cannula 404, and bone filler material (e.g., PMMA) can be delivered into cavity 402-V. As shown in FIG. 4G, a delivery nozzle 453 can be inserted through cannula 404 and into cavity 402-V, and can be used to direct bone filler material 455 into cavity 402-V. In some embodiments, a quantity of bone filler material 455 can be housed in a cartridge 452 attached to delivery nozzle 453. A hydraulic actuator 450 can then be used to remotely express bone filler material 455 from cartridge 452 via a hydraulic line 451 (e.g., cartridge 452 can include a piston that is driven by the hydraulic pressure supplied by hydraulic line 451).

Note, however, that in various other embodiments, bone filler material 455 can be delivered to cavity 402-V in any number of different ways (e.g., a high pressure cement delivery pump that delivers the cement to nozzle 453 through a flexible line, or a syringe or other delivery device filled with bone filler material 455 that is attached directly to nozzle 453, or even directly to cannula 404). In addition, in various other embodiments, bone filler material 455 can be delivered in multiple portions of the same or different materials (e.g., a bone cement followed by a biologic agent).

Once the filling operation is complete, delivery nozzle 453 and cannula 404 are removed from vertebra 402 (and the patient's body) as shown in FIG. 4H. Upon hardening, bone filler material 455 provides structural support for vertebra 402, thereby substantially restoring the structural integrity of the bone and the proper musculoskeletal alignment of the spine. As shown in FIG. 4H, due to the restoration of height in fractured vertebra 402, the abnormal curvature CK shown in FIG. 4A is corrected to a normal curvature CN. In this manner, the pain and attendant side effects of a vertebral compression fracture can be addressed by a minimally invasive kyphoplasty procedure.

Note that although a kyphoplasty procedure is depicted and described for exemplary purposes, inflatable bone tamp 100 can be similarly used in any other target surgical location in or around bone, such as a tibial plateau fracture, a proximal humerus fracture, a distal radius fracture, a calcaneus fracture, a femoral head fracture, among others. For example, to restore a tibial plateau fracture, the working length of inflatable structure 110 could be decreased to provide more localized lifting (e.g., for a Type I fracture), or could be increased to provide a larger lift surface area (e.g., for a Type III fracture). Various other usages will be readily apparent.

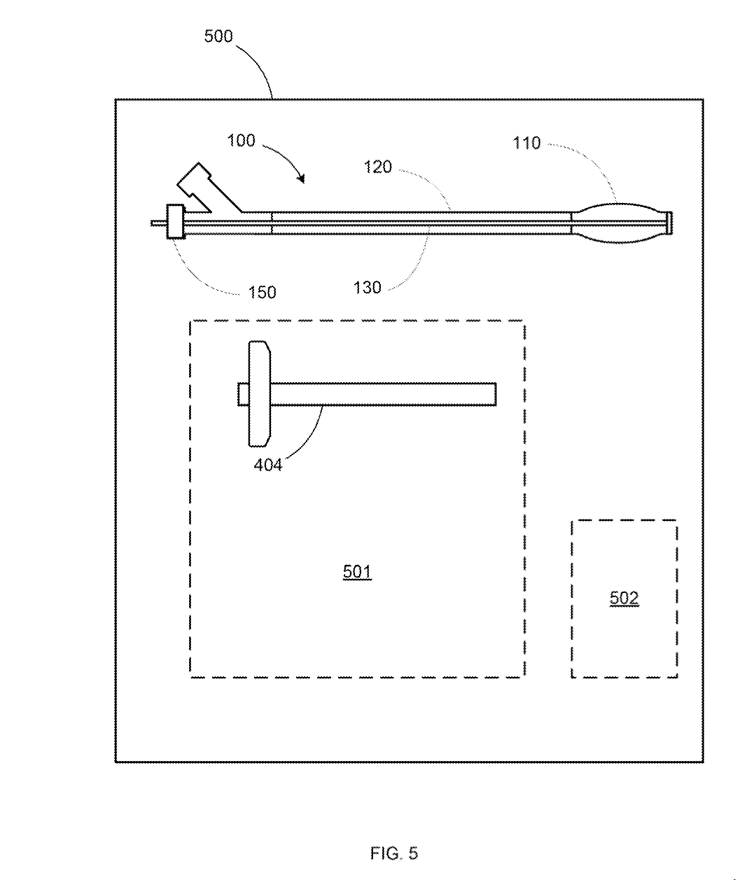

FIG. 5 shows a diagram of a kit 500 for use in performing a surgical procedure, such as a kyphoplasty procedure described with respect to FIGS. 4A-4H above. Kit 500 includes an inflatable bone tamp 100 (e.g., as described above with respect to FIGS. 1A-1B and 4A-4H) having an inner shaft 130 positionally controlled by an extension controller 150. In various embodiments, kit 500 can further include optional additional instruments 501, such as a cannula 404 sized to receive inflatable bone tamp 100, an introducer, guide pin, drill, curette, and/or access needle, among others (only cannula 404 is shown for clarity). In various other embodiments, kit 500 can further include optional directions for use 502 that provide instructions for using inflatable bone tamp and optional additional instruments 501 (e.g., instructions for performing a kyphoplasty procedure using inflatable bone tamp 100 and optional additional instruments 501 as described with respect to FIGS. 4A-4H).

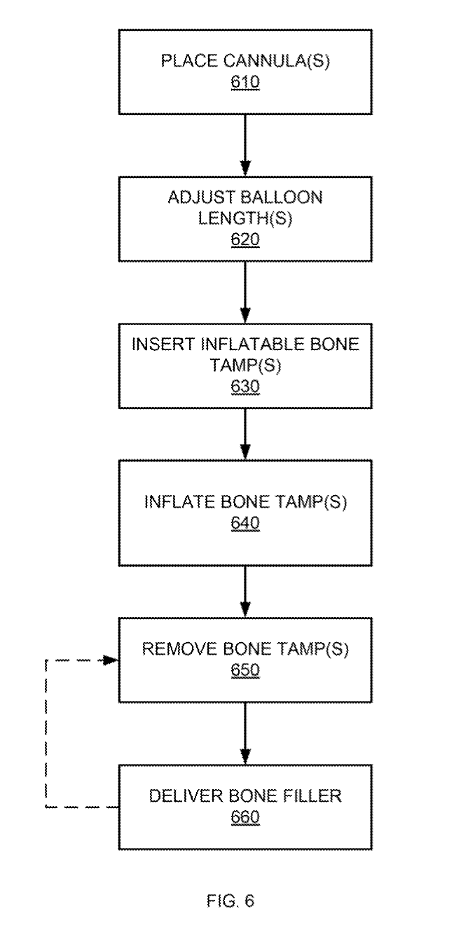

FIG. 6 shows a flow diagram of a process for performing a surgical procedure such as kyphoplasty using an inflatable bone tamp including an inflatable bone tamp that allows for adjustment of balloon working length, as described with respect to FIGS. 1A-1B. In a PLACE CANNULA(S) step 610, a cannula is positioned within a patient to provide a path to a target surgical location (e.g., as described with respect to FIG. 4B). Note that although a unilateral procedure is described above for clarity, in various other embodiments, a bilateral procedure can be used (e.g., placing two cannulas to provide access through both pedicles of a vertebra).

I an ADJUST BALLOON LENGTH(S) step 620, the working length(s) of the inflatable structure(s) (e.g., inflatable structure 110) are increased/decreased as described with respect to FIGS. 1A-1B, 2A-2B, and 4C. Note that according to various embodiments, step 620 can be performed prior to, simultaneously with, and/or after step 610.

Then, in an INSERT INFLATABLE BONE TAMP(S) step 630, the inflatable bone tamp(s) is placed within the patient through the cannula (e.g., as described with respect to FIG. 4D). Note once again that if multiple cannulas have been placed in step 610, an inflatable bone tamp can be inserted into each cannula.

Next, in an INFLATE BONE TAMP(S) step 630, the inflatable bone tamp(s) is (are) inflated to create a cavity in cancellous bone and, ideally, at least partially restore the original cortical bone profile (e.g., as described with respect to FIG. 4E). As described above, the balloon length set in step 620 can optimize the performance of the inflatable bone tamp during step 640. Note that if multiple inflatable bone tamps have been introduced in step 630, their inflation can be sequential, simultaneous, sequentially incremental (e.g., partially inflating one before partially or fully inflating another), or any other order.

The inflatable bone tamp is then deflated and withdrawn from the patient in a REMOVE BONE TAMP(S) step 650 (e.g., as described with respect to FIG. 4F), and in a DELIVER BONE FILLER step 660, a bone filler material (e.g., bone cement) is conveyed to the cavity formed by the inflatable bone tamp to create a permanent reinforcing structure within the bone (e.g., as described with respect to FIGS. 4G and 4H).

Note that if multiple bone tamps have been placed within the patient (e.g., in a bilateral procedure) in step 620, one or more of those inflatable bone tamps can be left (inflated) within the patient to provide support for the bone structure during subsequent material delivery during step 660. The process can then loop back to step 650 and then step 660 until all inflatable bone tamps have been removed, and all the resulting cavities in the bone have been filled with bone filler material.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

1. A device for performing a surgical procedure, the device comprising:
an outer shaft;
an inner shaft disposed within the outer shaft;
an inflatable structure having a proximal end coupled to a distal end of the outer shaft and a distal end coupled to a distal end of the inner shaft; and
an extension controller for adjusting and securing a relative position between the distal end of the inner shaft and the distal end of the outer shaft.

2. The device of claim 1, wherein the extension controller comprises at least one of a ratchet, a clamp, and a latch for securing the relative position between the distal end of the inner shaft and the distal end of the outer shaft.

3. The device of claim 1, wherein the extension controller comprises a pull roller.

4. The device of claim 3, wherein the inner shaft comprises at least one of a catheter, a stainless steel wire, and a nitinol wire.

5. The device of claim 1, wherein the extension controller comprises at least one of a spur gear, a helical gear, a worm wheel gear, and a rack gear.

6. The device of claim 5, wherein the inner shaft comprises a series of features for interfacing with the at least one of the spur gear, the helical gear, the worm wheel gear, and the rack gear.

7. The device of claim 1, further comprising:
a connector coupled to the outer shaft defining a delivery path for inflation fluid to be delivered to the inflatable structure; and
a sealing element for sealing around the inner shaft to prevent leakage of the inflation fluid from around the inner shaft.

8. The device of claim 1, wherein the sealing element comprises a Tuohy-Borst connector.

9. The device of claim 1, further comprising a rotation controller for rotating the inner shaft relative to the outer shaft to wrap the inflatable structure around the inner shaft.

10. A surgical kit comprising:
a cannula defining an access lumen; and
an inflatable bone tamp sized to pass through the access lumen, the inflatable bone tamp comprising:
an outer shaft;
an inner shaft disposed within the outer shaft;
an inflatable structure coupled between a distal end of the outer shaft and a distal end of the inner shaft; and
an extension controller for adjustably setting a relative position between the inner shaft and the outer shaft to define a working length of the inflatable structure.

11. The system of claim 10, wherein the extension controller comprises at least one of a ratchet, a clamp, and a latch for securing the relative position between the inner shaft and the outer shaft.

12. The system of claim 10, wherein the extension controller comprises a pull roller, a spur gear, a helical gear, a worm wheel gear, and a rack gear.

13. The system of claim 10, wherein the inflatable bone tamp further comprises:
a connector coupled to the outer shaft defining a delivery path for inflation fluid to be delivered to the inflatable structure; and
a sealing element to prevent leakage of the inflation fluid from around the inner shaft.

14. The system of claim 10, wherein the inflatable bone tamp further comprises a rotation controller for rotating the inner shaft relative to the outer shaft to wrap the inflatable structure around the inner shaft.

15. A method comprising:
creating an access path to a bone structure comprising cancellous bone;
providing an inflatable bone tamp comprising an outer shaft, an inner shaft disposed within the outer shaft, and an inflatable structure coupled between the outer shaft and the inner shaft;
adjusting a relative position between the inner shaft and the outer shaft;
securing the relative position between the inner shaft and the outer shaft to define a working length for the inflatable structure;
inserting the inflatable bone tamp into the access path to position the inflatable structure within the bone structure; and
inflating the inflatable structure to compress a portion of the cancellous bone and create a cavity.

16. The method of claim 15, wherein adjusting a relative position between the inner shaft and the outer shaft comprises rotating an actuator to move the inner shaft relative to the outer shaft.

17. The method of claim 15, wherein securing the relative position between the inner shaft and the outer shaft comprises engaging at least one of a ratchet, a latch, and a clamp.

18. The method of claim 15, further comprising:
removing the inflatable bone tamp from the access path; and
delivering a bone filler material into the cavity through the access path.

19. The method of claim 18, wherein creating the access path comprises docking a cannula with the bone structure, and
wherein delivering the bone filler material comprises inserting a delivery nozzle into the cannula and injecting the bone filler material into the cavity from the delivery nozzle.

20. The method of claim 18, wherein removing the inflatable bone tamp from the access path comprises rotating the inner shaft to wrap the inflatable structure around the inner shaft.
